(19) United States
(12) Patent Application Publication
(10) Pub. No.: US 2026/0351364 A1
(43) Pub. Date: Nov. 5, 2026

(54) SYSTEM AND METHOD FOR THE ANALYSIS OF MEDICAL INFORMATION FOR SCORING MEDICAL TREATMENTS

(71) Applicant: Sutherland Global Services Inc., Pittsford, NY (US)

(72) Inventor: John Paul Backhouse, Valparaiso, IN (US)

(73) Assignee: Sutherland Global Services Inc., Pittsford, NY (US)

(21) Appl. No.: 19/666,653

(22) Filed: May 4, 2026

Related U.S. Application Data
(60) Provisional application No. 63/798,765, filed on May 2, 2025.

(51) Int. Cl. G16H 10/60; G16H 15/00

(52) U.S. Cl. CPC G16H 10/60; G16H 15/00

(57) ABSTRACT

Methods and computer program products for scoring medical treatments are provided. The method includes training a large concept model (LCM) based on labeled electronic health record (EHR) data and a selected training algorithm to generate a trained LCM. A first score representing the severity of an association between a medical condition and a symptom and/or biomarker of EHR data is determined using the trained LCM. The EHR data is associated with a patient. A second score representing the severity of an association between a diagnostic test and the medical condition is determined using the trained LCM. A third score representing the severity of an association between a medical treatment and the medical condition is determined using the trained LCM. The first score, the second score, and/or the third score are stored in association with the EHR data.

### CROSS-REFERENCE TO RELATED APPLICATIONS

This application claims the benefit of U.S. Patent Application No. 63/798,765, filed May 2, 2025, the contents of which are incorporated by reference herein in its entirety.

### FIELD OF THE INVENTION

The present disclosure relates generally to the field of medical information processing, and more particularly to the analysis of medical information for scoring medical treatments.

### BACKGROUND OF THE INVENTION

Medical care inefficiencies significantly burden healthcare systems, which can drive up costs, delay treatments, and/or compromise patient outcomes. Fragmented patient data across electronic health record (EHR) systems often leads to poor interoperability, making it difficult for clinicians to access comprehensive patient histories in real time. Administrative tasks can contribute to errors, delays, and inefficiencies such as redundant tests or manual processes. Consumers would benefit from solutions that reduce fragmentation of patient data, errors, delays, redundant tests, and/or diagnostic errors. The present invention fills these needs as well as other needs.

### SUMMARY OF THE INVENTION

Briefly described, the present disclosure is directed to a selection of concepts in a simplified form which are further described below. This summary is not intended to identify key features or essential features of the claimed subject matter, nor is it intended to be used as an aid in determining the scope of the claimed subject matter. The claimed subject matter is not limited to implementations that solve any or all disadvantages noted in the background.

In view of the foregoing, methods and computer program products for scoring medical treatments are provided. In one general aspect, the method may include training, by a computing device, a large concept model (LCM) based on labeled electronic health record (EHR) data and a selected training algorithm to generate a trained LCM. The selected training algorithm can include a backpropagation algorithm, an optimization algorithm, a forward propagation algorithm, an initialization algorithm, a regularization algorithm, a gradient boosting tree algorithm, and/or a masked language modeling algorithm. The method may also include determining, by the computing device and using the trained LCM, a first score representing a severity of an association between a medical condition and a symptom and/or a biomarker included in EHR data. The EHR data is associated with a patient. The method may furthermore include determining, by the computing device, a second score representing a severity of an association between a diagnostic test and the medical condition using the trained LCM.

The method may in addition include determining, by the computing device, a third score representing a severity of an association between a medical treatment and the medical condition using the trained LCM. The method may moreover include storing, in a non-transient data store, the first score, the second score, and/or the third score in association with the EHR data. The non-transient data store can include a vector database. The EHR data can be stored in a second vector database. The EHR data can include information generated in real time.

The method may also include generating, by the computing device, a report based on the EHR data using the trained LCM. The report can include a consultation report, a diagnostic report, a discharge summary, a medico-legal report, and/or a progress report. The method can also include receiving, by the computing device, user authentication data and determining, by the computing device, whether authenticated user data comprises the user authentication data. Here, the step of determining the first score can include determining the first score when the authenticated user data is confirmed to include the user authentication data. In some aspects, the method can further include generating, by the computing device, a user notification that includes an identified medical condition associated with the first score when the first score is at least a first predetermined value, an identified diagnostic test associated with the second score when the second score is at least a second predetermined value, and/or an identified medical treatment associated with the third score when the third score is at least a third predetermined value.

In certain general aspects, a computer program product includes a computer readable storage medium having program code embodied therewith, the program code executable by a processor to may include program code to train a LCM based on labeled EHR data and a selected training algorithm to generate a trained LCM; determine a first score representing a severity of an association between a medical condition and a symptom and/or a biomarker included in EHR data using the trained LCM; determine a second score representing a severity of an association between a diagnostic test and the medical condition using the trained LCM; determine a third score representing a severity of an association between a medical treatment and the medical condition using the trained LCM; and store, in a non-transient data store, the first score, the second score, and/or the third score in association with the EHR data.

In one general aspect, a method for scoring medical treatments may include determining a feature to extract from EHR data. The feature represents a medical parameter of the EHR data. The EHR is associated with a patient. The method may also include extracting the feature from the EHR data to form feature data and processing the feature data using a machine learning model (MLM) trained with labeled EHR data. The labeled EHR data can represent instances of (i) a plurality of symptoms and/or a plurality of biomarkers each associated with at least one of a plurality of medical conditions, (ii) a plurality of diagnostic tests each associated with at least one of said medical conditions, and/or a (iii) plurality of medical treatments each associated with at least one of said medical conditions.

The labeled EHR data associating (i) each of a plurality of first scores with a severity of an indicative association between one of said medical conditions and at least one of said symptoms and/or at least one of said biomarkers, (ii) each of a plurality of second scores with a severity of an identifying association between one of said diagnostic tests and at least one of said medical condition, and/or (iii) and each of a plurality of third scores with a severity of a managerial association between one of said medical treatments and at least one of said medical condition.

The step of processing the feature data can include the following: generating a semantic representation corresponding to the EHR data; determining, based on the processing and the semantic representation, a first determined score, a second determined score, and/or a third determined score; and storing, in a non-transient data store, the first determined score, the second determined score, and/or the third determined score in association with the EHR data. The first determined score can correspond to one of said first scores. The second determined score can correspond to one of said second scores. The third determined score can correspond to one of said third scores.

### BRIEF DESCRIPTION OF THE DRAWINGS

The above-mentioned and other features and advantages of this invention, and the manner of attaining them, will become apparent and be better understood by reference to the following description of the invention in conjunction with the accompanying drawing, wherein:

FIG. 1 is a block diagram illustrating a medical information processing environment, in accordance with some embodiments of the instant disclosure;

FIG. 2 is a block diagram illustrating a large concept model training pipeline architecture, in accordance with certain embodiments of the instant disclosure;

FIG. 3 is a flowchart depicting operational steps of a program for scoring medical treatments , in accordance with other embodiments of the instant disclosure;

FIG. 4 is a flowchart depicting operational steps of a patient management program, in accordance with yet still other embodiments of the instant disclosure;

FIG. 5 is a block diagram illustrating a data processing system, in accordance with an illustrative embodiment of the instant disclosure; and

FIG. 6 illustrates an example deep neural network in which embodiments of the present technology may be implemented.

Corresponding reference characters indicate corresponding parts throughout the several views. The exemplifications set out herein illustrate currently preferred embodiments of the present invention, and such exemplifications are not to be construed as limiting the scope of the invention in any manner.

### DETAILED DESCRIPTION OF THE EMBODIMENTS

As will be appreciated by one skilled in the art, aspects of the instant disclosure may be embodied as a system, method or computer program product. Accordingly, aspects of the instant disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the instant disclosure may take the form of a computer program product embodied in one or more computer-readable medium(s) having computer-readable program code/instructions embodied thereon.

Any combination of computer-readable media may be utilized. Computer-readable media may be a computer-readable signal medium or a computer-readable storage medium. A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of a computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, or any suitable combination thereof. A computer-readable signal medium may be any computer-readable medium that is not a computer-readable storage medium and that can communicate, propagate, or transport a program for use by or in connection with an instruction execution system, apparatus, or device. Program code embodied on a computer-readable medium may be transmitted using any appropriate medium, including but not limited to wireless, wireline, optical fiber cable, RF, etc., or any suitable combination of the foregoing.

Computer program code for carrying out operations for aspects of the instant disclosure may be written in any combination of one or more programming languages, including an object oriented programming language such as Java, Smalltalk, C++ or the like and conventional procedural programming languages, such as the "C" programming language or similar programming languages. The program code may execute entirely on a user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the instant disclosure are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer-readable medium that can direct a computer, other programmable data processing apparatus, or other devices to function in a particular manner, such that the instructions stored in the computer-readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer-implemented process such that the instructions which execute on the computer or other programmable apparatus provide processes for implementing the functions/acts specified in the flowchart and/or block diagram block or blocks.

The terminology used herein is for the purpose of describing particular aspects only and is not intended to be limiting of the disclosure. As used herein, the singular forms "a," "an" and "the" are intended to include the plural forms as well, unless the context clearly indicates otherwise. It will be further understood that the terms "comprises" and/or "comprising," when used in this specification, specify the presence of stated features, integers, steps, operations, elements, and/or components, but do not preclude the presence or addition of one or more other features, integers, steps, operations, elements, components, and/or groups thereof.

The description of the present disclosure has been presented for purposes of illustration and description but is not intended to be exhaustive or limited to the disclosure in the form disclosed. Many modifications and variations will be apparent to those of ordinary skill in the art without departing from the scope and spirit of the disclosure. The aspects of the disclosure herein were chosen and described in order to best explain the principles of the disclosure and the practical application, and to enable others of ordinary skill in the art to understand the disclosure with various modifications as are suited to the particular use contemplated.

For purposes of this document, each process associated with the disclosed technology may be performed continuously and by one or more computing devices. Each step in a process may be performed by the same or different computing devices as those used in other steps, and each step need not necessarily be performed by a single computing device.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

The present disclosure relates generally to the field of medical information processing, and more particularly to the analysis of medical information for scoring medical treatments. Medical care inefficiencies significantly burden healthcare systems, driving up costs, delaying treatments, and compromising patient outcomes. Fragmented patient data across electronic health record (EHR) systems often leads to poor interoperability, making it difficult for clinicians to access comprehensive patient histories in real time. Administrative tasks can contribute to errors, delays, and inefficiencies such as redundant tests or manual processes.

The instant disclosure aims to provide AI models that can mitigate inefficiencies by integrating and analyzing EHR data to provide actionable insights. The instant disclosure seeks to provide large concept models (LCM) which are specialized deep neural networks—typically transformer-based—that are configured to focus on learning and reasoning over abstract concepts. An LCM is a type of language model that processes language at the concept level rather than analyzing individual words. Unlike traditional models that break down text word by word, LCMs interpret semantic representations, which correspond to entire sentences or cohesive ideas. The instant disclosure seeks provide LCMs that are configured to comprehend the broader meaning of language included in EHR data rather than just the mechanics thereof.

Embodiments of the instant disclosure seek to provide trained LCMs (TLCM) to synthesize complex EHR data and thereby generate scores representing associations between medical conditions and symptoms and/or biomarkers, scores representing associations between diagnostic tests and medical conditions, and scores representing associations between medical treatments and medical conditions.

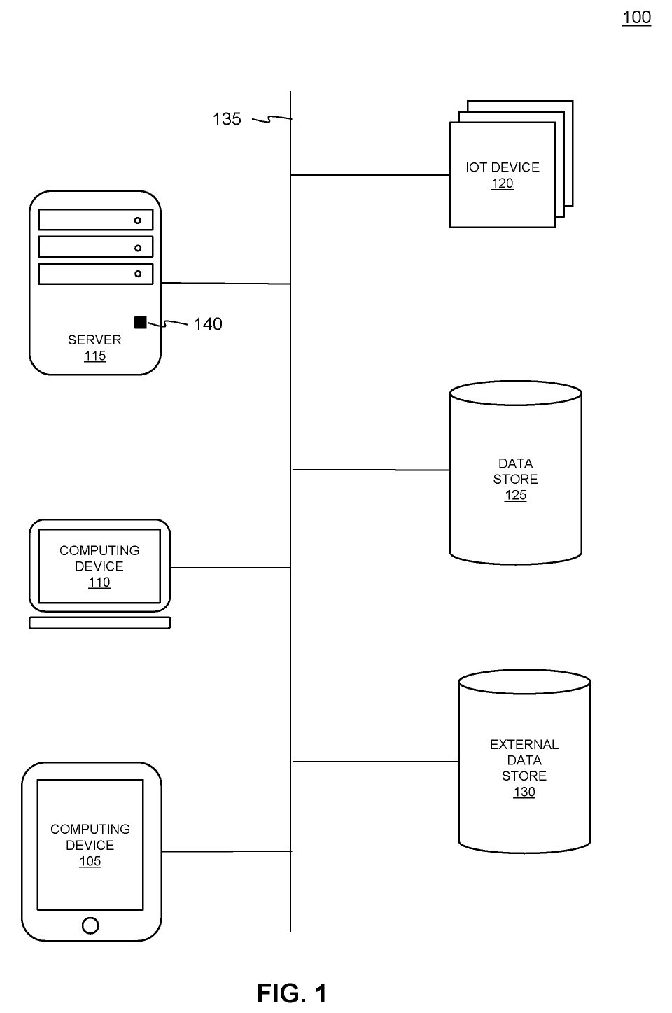

Embodiments of the instant disclosure will now be described in detail with reference to the FIGS. FIG. 1 is a block diagram illustrating a medical information processing environment, generally designated 100, in accordance with some embodiments. Environment 100 includes computing devices 105 and 110, server 115, internet of things (IoT) device(s) 120, data store 125, and external data store 130, all interconnected over network 135. Network 135 can be, for example, a local area network (LAN), a wide area network (WAN) such as the Internet, or a combination of the two, and can include wired, wireless, or fiber optic connections. In general, network 135 can be any combination of connections and protocols that will support communications between computing devices 105 and 110, server 115, IoT device(s) 120, data store 125, and/or external data store 130.

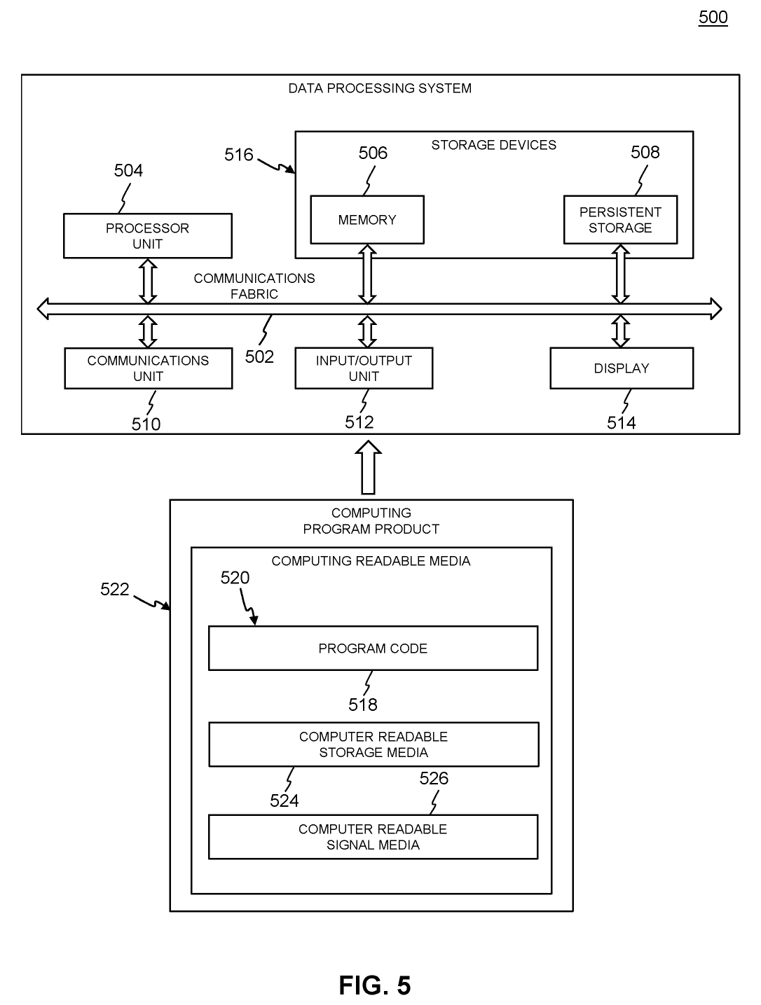

In various embodiments, computing devices 105 and 110, and/or server 115 may each be a laptop computer, tablet computer, netbook computer, personal computer (PC), desktop computer, personal digital assistant (PDA), mobile computing device, or any programmable electronic device capable of communicating with a computing device and/or data store via network 135. Server 115 may be one or more computing devices that executes one or more of the process steps and/or language models described herein. Server 115 can be a tool used to collect data as well as train, test, and tune ML models based on the collected data (e.g., labeled EHR data and/or training data). Server 115 can include program files 140. Computing devices 105 and 110, and/or server 115 may include internal and external hardware components, as depicted and described in further detail with respect to FIG. 5.

To be sure, IoT device 120 can be one or more computing devices that connect to the internet and/or network 135 and interact with other devices connected thereto, allowing for remote management and control. These devices are preferably integrated with high-definition technology, including sensors, functional software, and network connectivity, enabling them to communicate seamlessly over the internet. For example, IoT device 120 can be an electronic medical device that provides real time monitoring of patient vitals including, but not limited to, body temperature, heart rate, respiration rate, and blood pressure, and/or oxygen saturation. IoT device 120 can enable continuous home (i.e., distant) patient monitoring. In certain embodiments, IoT device 120 can be accessed by a user using a computing device communicatively coupled to network 135 (e.g., computing device 105 and/or 110). Data generated by IoT device 120 (i.e., EHR data) can be stored internally and/or in data store 125 as well as other data stores communicatively coupled to network 135. Such data is preferably stored in a format compliant with the Health Insurance Portability and Accountability Act (HIPPA) or other regulations.

Data store 125 and external data store 130 can each be one or more repositories for persistently storing and managing collections of data (e.g., EHR data, labeled EHR data, training data, or other data). For example, data store 125 (or other data store communicatively coupled to network 135) can include a vector database that stores, manages, and queries data as high-dimensional vectors (e.g., numerical representations of data like text, images, or audio) enabling efficient similarity searches based on vector proximity. Such vector databases are specialized data systems designed to enable semantic search by storing and querying information as mathematical representations known as vector embeddings. Unlike traditional keyword-based search, which depends on exact text matches, semantic search interprets the context and intent behind a query, allowing it to return more meaningful results. To achieve this, unstructured data such as text, images, or audio is first processed by a machine learning model—such as bidirectional encoder representations from transformers (BERT) primarily designed for understanding context or embedding models (that can convert text into numerical vectors to measure relatedness, which are then used in search, clustering, and recommendations)—which converts the data into a high-dimensional vector of numbers. These vectors are then placed within a multi-dimensional vector space, where items with similar meanings are positioned closer together; for example, the concepts “shortness of breath” and “pneumonia” would appear near one another.

When a user submits a query, it is also transformed into a vector and compared against the stored vectors using similarity metrics (e.g., cosine similarity, Euclidean distance, or other similarity metrics) to identify the most relevant matches. To maintain fast performance at scale, vector databases rely on Approximate Nearest Neighbor (ANN) algorithms rather than computing distances against every stored vector. Techniques such as Hierarchical Navigable Small World (HNSW) enable the system to efficiently locate the closest vectors, balancing accuracy and speed even when working with very large datasets.

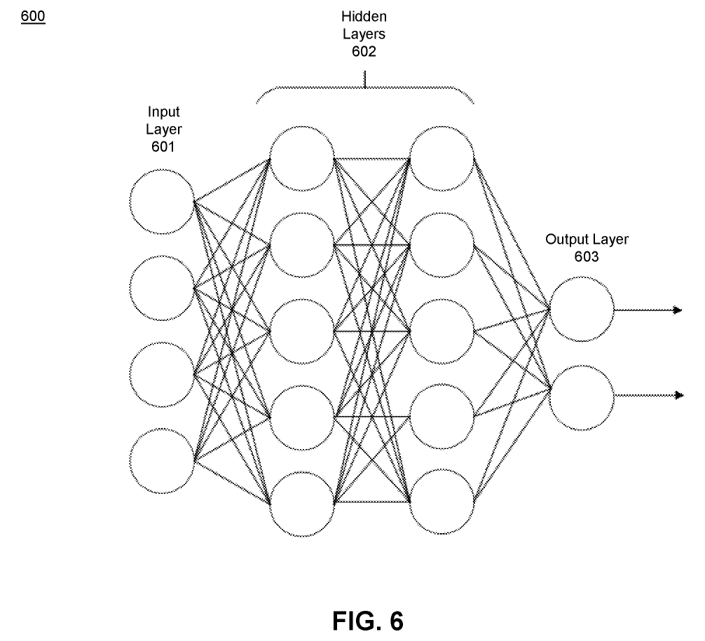

The instant disclosure seeks to provide LCMs (e.g., at least one TLCM) that can process EHR data and generate scores related thereto. In certain embodiments, applicable LCMs can have one or more aspects, elements, and/or components of deep neural network 600 (see FIG. 6 discussion below). Program files 140 can include one or more aspects of the LCMs, TLCM, and/or other language models. Program files 140 can include software components that collect training data sets (e.g., labeled EHR data and labeled scientific data, and/or labeled medical data) and generates trained models.

Although large language models (LLMs) are powerful tools, they often struggle with keeping track of big-picture ideas. That is because LLMs work by predicting text one token, or word, at a time. This token-by-token approach, combined with a limited context window, can lead to disjointed responses, lost context, and lots of repetition. This approach is similar to drafting an essay by guessing each next word instead of outlining your thoughts first. This is where LCMs have an advantage. Instead of working word by word, LCMs can process language at the sentence level and abstract language into concepts. This abstraction allows the model to understand language in a more thoughtful and meaningful way. An LCM is a type of language model that processes language at the concept level rather than analyzing individual words. Unlike traditional models that break down text word by word, LCMs interpret semantic representations, which correspond to entire sentences or cohesive ideas. This shift allows them to grasp the broader meaning of language rather than just the mechanics and therefore preferred for analyzing EHR data.

Generative AI model can refer to learning models that can create new and original content or data by thoroughly analyzing and learning from established patterns found in historical information (e.g., training data). In other embodiments, program 140 can include generative AI models that are configured to automate the creation of patient summaries, detailed medical histories, and comprehensive clinical notes, which can reduce the administrative workloads faced by healthcare clinicians. The generative AI models can analyze extensive datasets to customize treatments based on individual patient profiles (e.g., included in the EHR data), significantly enhancing therapeutic outcomes and providing a more effective approach to patient care. The generative AI models can create realistic and diverse patient data that can be utilized for the training and validation of other AI models, significantly enhancing compliance with data privacy regulations and protections.

In contrast, agentic AI models can refer to language models that focus on autonomous systems that make decisions and perform tasks without human intervention. The independent systems automatically respond to conditions, to produce process results. Unlike traditional AI, which operates within predefined constraints, agentic AI thrives on adaptability and continuous improvement. Agentic AI differs from traditional AI models in its ability to autonomously take actions rather than simply responding to prompts, iterate and self-improve through reinforcement learning, break down complex tasks into subtasks and execute them in sequence, and collaborate with other agents in a distributed computing environment.

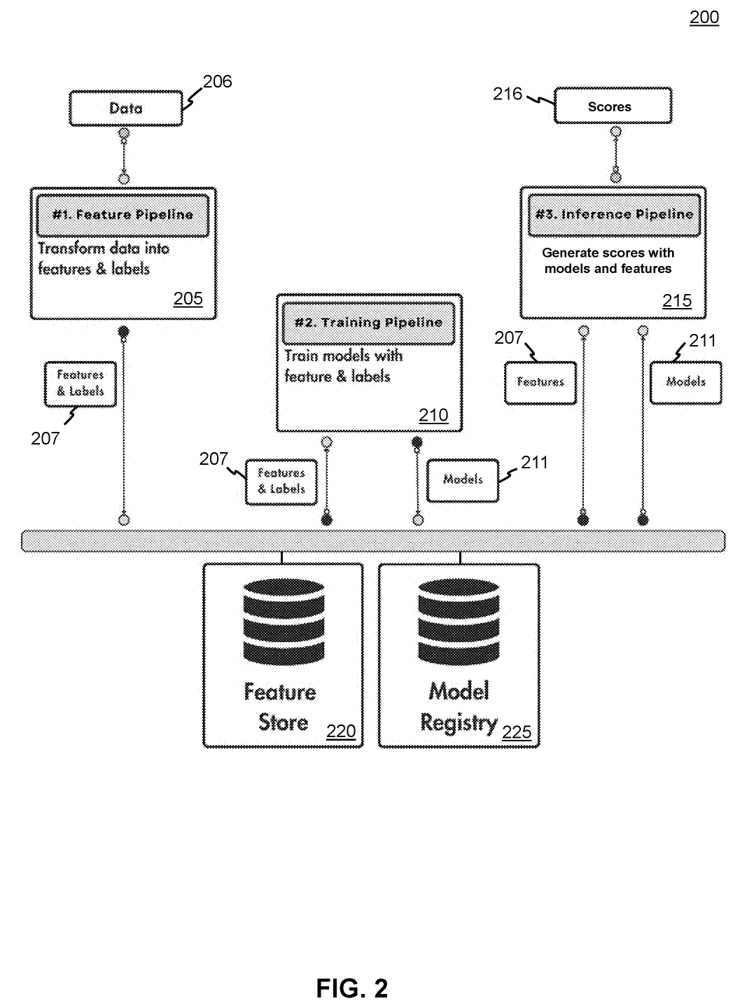

FIG. 2 is a block diagram of an LCM training pipeline architecture, generally 200, according to certain embodiments. As used herein, a “pipeline” refers to the process steps required to achieve a particular task. Program files 140 may include one or more of said process steps. Training pipeline architecture 200 is based on feature pipeline 205, training pipeline 210, and inference pipeline 215. Feature pipeline 205 takes data 206 (e.g., training data stored in data store 125 and/or external data store 130) as input, processes it, and outputs features and labels 207 required by the model for training or inference. For example, data 206 can include, but is not limited to, medical journal articles, scientific/medical research data, labeled EHR data, clinical trial data, clinical notes, IoT device generated data, medical images and/or other relevant data. Generated features and labels 207 may be stored inside feature store 220.

Next, training pipeline 210 can utilize features and labels 207 from feature store 220 as inputs and generate one or more trained models 211 (e.g., trained LCMs) that can be stored in model registry 225. In some embodiments, model registry 225 can be stored in server 115, data store 125, and/or other data stores communicatively coupled to network 135. Model registry 225 can store, version, track, and/or share model 211 with inference pipeline 215. Lastly, inference pipeline can use features and labels 207 (e.g., derived from patient EHR data) from feature store 220 as input and trained model 211 from model registry 225 to generate scores in either batch or real-time mode. Such scores can include scores representing associations between medical conditions and symptoms and/or biomarkers identified in the EHR data, scores representing associations between diagnostic tests and the medical conditions, and scores representing associations between medical treatments and the medical conditions. Training pipeline 210 use high data volume, complex calculations, real-time processing, and/or dynamic updates to generate trained models 211.

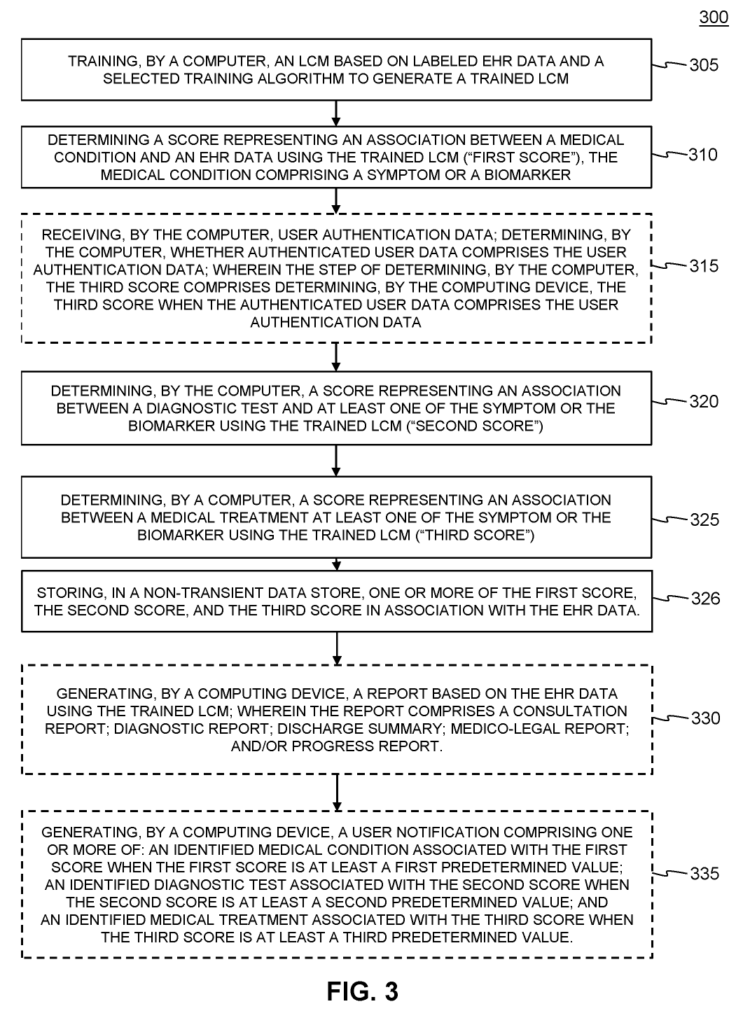

FIG. 3 is a flowchart depicting operational steps of a program for scoring medical treatments (scoring program), generally 300, in accordance with certain embodiments. Scoring program 300 is software that can use a trained LCM to score medical treatments. Program files 140 can include one or more operational steps of scoring program 200, which may be executed via one or more computer processors. At Step 305, an LCM is trained at least on labeled EHR data and/or other training data (e.g., data 206) and a selected training algorithm to thereby generate a trained LCM (TLCM). For example, the LCM can be trained using pipeline architecture 200. EHR data and/or the labeled EHR data can be stored in data store 125 and/or external data store 130. The selected training algorithm can include, but is not limited to, a backpropagation algorithm, optimization algorithm, forward propagation algorithm, initialization algorithm, regularization algorithm, gradient boosting tree algorithm, and/or masked language modeling algorithm.

At Step 310, a first score representing a severity of an association between a medical condition and a symptom and/or a biomarker included in the EHR data is determined using the trained LCM. The EHR data is associated with a patient. The EHR data can include information generated in real time—that is, information that is delivered, processed, and/or made available for analysis immediately after it is generated or collected. Real-time data are characterized by minimal latency, typically within milliseconds to seconds, enabling systems and users to respond to events as they occur. In certain embodiments, the EHR data can include data generated by a speech-to-text algorithm. For example, medical conditions are typically associated with (i.e., indicative of) one or more symptoms and/or biomarkers. As such, in some embodiments, the first score represents the severity of an indicative association between the medical condition and the symptom and/or the biomarker. In other words, the first score can reflect how strongly the symptom and/or the biomarker indicates the presence of the associated medical condition. For example, in the case of diabetes, a high first score would indicate a strong association between the condition and symptoms such as frequent urination and thirst, as well as biomarkers like elevated HbA1c levels. To be sure, scores can reflect proportional or inverse relationships. Here, the TLCM can interpret information included in the EHR data to ascertain the symptoms (e.g., frequent urination and thirst).

Scoring program 300 can also be configured to address privacy concerns. For example, EHR data typically contains regulated data because it includes sensitive, identifiable information about a patient’s health and care. This can include protected health information such as patient identifiers, clinical notes, diagnoses, laboratory and diagnostic results, medications, treatments, and behavioral or mental health records. EHR data may also include administrative and billing information, device-generated data, and demographic or social determinants of health, all of which are regulated to protect patient privacy and safety.

Given that EHR data can include regulated data and/or sensitive data, access to the EHR data can be restricted to authorized users. For example, at Step 315, user authentication data is received, and a determination is made as to whether stored authenticated user data includes the received user authentication data. User authentication data can include, but is not limited to, passwords, biometric data, and/or other identifying data. Authenticated user data (i.e., data determined to be associated with users authorized to access at least a portion of the EHR data) can be stored in data store 125 or other data stores communicatively coupled to network 135. Here, when the authenticated user data includes (e.g., includes data that matches) the user authentication data, the TLCM determines the first score, the second score, and/or third score. In general, scores above or below a predetermined value (e.g., indicating medical conditions of interest, diagnostic tests of interest, medical treatments of interest) can be isolated for further processing.

At Step 320, a second score representing an association between a diagnostic test and the medical condition is determined using the trained LCM. For example, diagnostic tests can identify or confirm the presence of medical conditions based on associated symptoms and/or biomarkers. As such, in certain embodiments, the second score can represent the severity of an identifying association between a diagnostic test and the medical condition. In other words, the second score can reflect the strength of the diagnostic test’s ability to identify or confirm the associated medical condition (e.g., based on the symptom and/or biomarker). Returning to the diabetes example, a high second score might indicate that a fasting glucose test or HbA1c test reliably identifies the medical condition when symptoms (such as excessive thirst) or biomarkers (such as elevated blood sugar levels) are present. Second scores above and/or below a predetermined value can be identified for further analysis or processing.

At Step 325, a third score representing an association between a medical treatment and the medical condition is determined using the trained LCM. For example, medical treatment typically refers to the management and care of a patient by healthcare professionals to cure, prevent, manage, or reduce the symptoms of (or biomarkers associated with) a medical condition. As such, the third score can represent the severity of the strength of a managerial association between a medical treatment and the medical condition. In other words, the third score measures how strongly a medical treatment is associated with addressing a specific medical condition (e.g., including curing, preventing, managing, and/or reducing its symptoms or associated biomarkers). For example, in terms of diabetes, a high third score might indicate that treatments such as insulin therapy or metformin are strongly associated with successfully managing the condition, helping control blood glucose levels and guide ongoing care. Third scores that are above or below a predetermined score value can be identified for further processing. In other embodiments, the first score, the second score, and/or the third score can be displayed in a user interface accessible to computing device 105 and/or 110. For example, the user interface can be stored in program file 140. At step 326, the first score, the second score, and/or the third score can be stored in a non-transient data store (e.g., data store 125 or other data store communicatively coupled to network 135) in association with the EHR data.

In certain embodiments, at Step 330, a report is generated based on the EHR data using the TLCM. For example, the report be a consultation report, diagnostic report, discharge summary, medico-legal report, and/or progress report. In other embodiments, at Step 335, a user notification is generated that includes an identified medical condition associated with the first score when the first score is at least a first predetermined value, an identified diagnostic test associated with the second score when the second score is at least a second predetermined value, and/or an identified medical treatment associated with the third score when the third score is at least a third predetermined value. The user notification can be displayed via a user interface (UI) available via server 115 and accessible via computing devices 105 and/or 110. In some embodiments, the UI can be

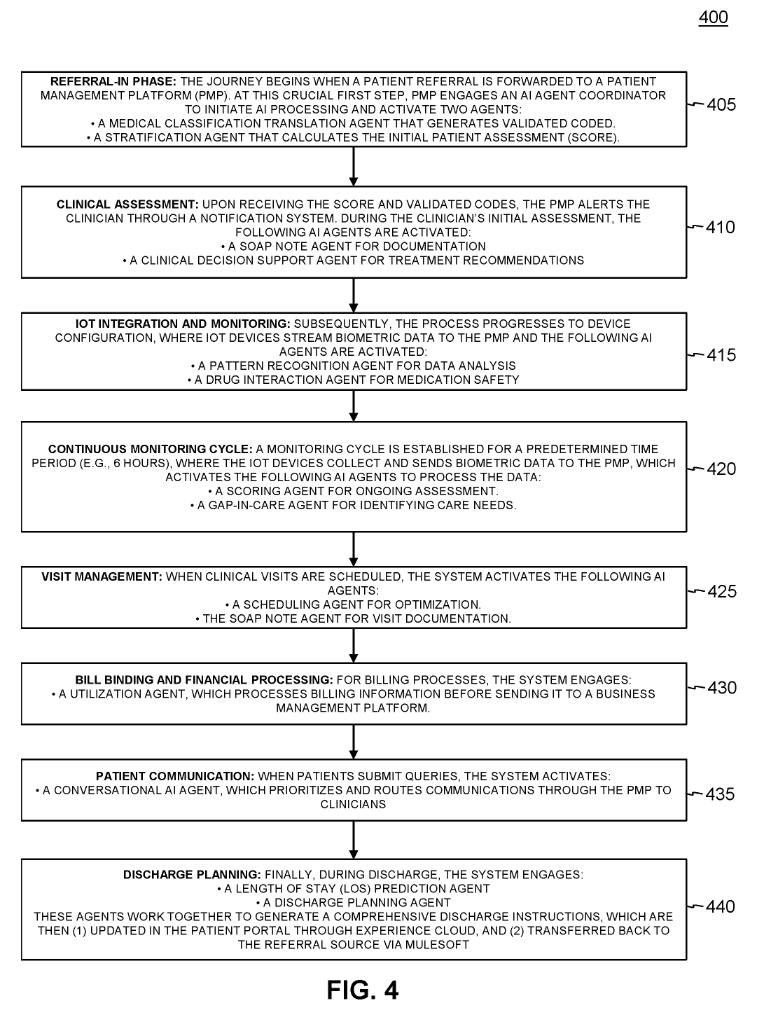

Alternatively, or in addition, the TLCM can be a plurality of AI agents working together to accomplish one or more tasks and/or process steps of scoring program 200. FIG. 4 is a flowchart, generally 400, depicting operational steps of a patient management program, in accordance with other embodiments of the instant disclosure. Patient management program 400 is software for an LCM having a plurality of AI agents collaborating to enable a seamless flow of information and care decisions from referral to discharge. Each agent's role can be precisely timed and integrated, ensuring comprehensive and coordinated patient care across all interface points in their healthcare journey. Patient management program 400 seeks to guarantee that every healthcare decision is informed by the current EHR data and AI-driven insights while maintaining smooth communication among all stakeholders in the patient's care journey.

At Step 405, the Referral-In Phase, a patient referral is forwarded (e.g., by a Referral Source) to a patient management platform (PMP) that subsequently engages an AI agent coordinator to initiate AI processing and activate a Medical Classification Translation Agent and Risk Stratification Agent. The Medical Classification Translation Agent can be configured to generate validated diagnosis codes and/or procedural codes. Applicable diagnosis codes and procedural codes include, but are not limited to, International Statistical Classification of Diseases and Related Health Problems (ICD) codes and Current Procedural Terminology (CPT) codes.

The Risk Stratification Agent can be configured to determine the initial patient assessment. The Stratification Agent can utilize gradient boosting trees (GBT), or similar machine learning technique, to generate a model(s) that analyzes patient data (e.g., inpatient EHR data) and generate scores. An applicable model can include, but are not limited to, long short-term memory (LSTM) or similar neural networks for modeling sequential data. Here, applicable patient data includes, but is not limited to, historical electronic health records (HER), lab results, vital signs (e.g., captured by IoT devices), social determinants, and/or claims data. In preferred embodiments, the Risk Stratification Agent has an AUROC (Area Under the Receiver Operating Characteristic Curve) score of at least 0.8. The Medical Classification Translation Agent can utilize a natural language processing (NLP) model, such as BERT, for code prediction.

At Step 410, subsequent to receiving the score and validated codes, the PMP transmits an alert to a clinician through a notification system. During the clinician’s initial assessment, a Clinical Note Agent and Clinical Decision Support Agent are activated. For example, the Clinical Note Agent can be configured to capture clinician notes (e.g., in the Subjective, Objective, Assessment, and Plan (SOAP) format or similar format). The Clinical Note Agent can utilize a text-to-text transformer model (e.g., T5) and/or Bidirectional Encoder Representations from Transformers (BERT) model for translation and/or summarization. Alternatively, or in addition, the Clinical Note Agent can use a BiLSTM (Bidirectional Long Short-Term Memory) or other type of recurrent neural network (RNN) architecture for NLP tasks, such as Named Entity Recognition (NER). The Clinical Note Agent training data can include, but is not limited to, clinical narratives, medical terminology, and/or template library.

The Clinician Decision Support Agent can be configured to provide treatment scores. For example, the Clinical Decision Support Agent can utilize a BERT model, probabilistic graphical model (e.g., Bayesian Networks), and deterministic system to generate severity scores in a healthcare context (e.g., patient outcomes, diagnosing diseases, HEDIS quality metrics). The Clinical Decision Support Agent can be trained using clinical guidelines, research literature, and/or practice patterns. The Clinical Decision Support Agent preferably has an accuracy of at least ninety percent (90%).

At Step 415, the process progresses to device configuration, where IoT devices stream data to the PMP, a Pattern Recognition Agent for data analysis, and Drug Interaction Agent for medication safety. For example, the Drug Interaction Agent can use Knowledge Graph Embeddings (KBE) and a transformer-based model (e.g., Molecular Transformer) to generate drug interaction severity scores. The Drug Interaction Agent can be trained using drug databases, drug interaction studies, and/or patient medications. In preferred embodiments, the Drug Interaction Agent can have a precision of at least 0.9.

At Step 420, a monitoring cycle is established for a predetermined (i.e., user determined) time period to allow the IoT devices to capture and transmit biometric data to the PMP for processing. Here, the PMP activates a Scoring Agent to analyze the data for ongoing assessments, and a Gap-in-Care Agent to identify care needs. At Step 425, when clinical visits are scheduled, a Scheduling Agent is activated for optimization as well as the Clinical Note Agent to capture visitation documentation. At Step 430, a Utilization Agent processes billing information and then sends it to a business management platform.

At Step 435, when a patient initiates communication (e.g., submits query), a Conversational AI Agent prioritizes and routes such communications through the PMP to the clinician. At Step 440, when the patient is discharged, a Length of Stay (LOS) Agent and Discharge Planning Agent are activated. These agents work together to generate comprehensive instructions, which are saved and subsequently transmitted to the Referral Source. For example, the LOS Agent can use a gradient-boosted tree algorithm, deep learning approach, and deep survival model to generate LOS scores. As used herein, LOS scores refer to estimating the duration a patient will remain hospitalized, which can be modeled as a time-to-event problem (time until discharge) or a regression problem (modeling LOS in days).

Applicable input data can include, but is not limited to, admission records, resource utilization data, staffing patterns data, and historical LOS data, which provides temporal and contextual information. For example, an ML algorithm (e.g., Extreme Gradient Boosting with temporal features or similar algorithm) can be used to process structured and temporal features from the training data to generate LOS scores or extract features for downstream modeling.

Further, a hierarchical generative approach to survival analysis (e.g., deep survival analysis or similar approach) can be used to model LOS as a time-to-event outcome, accounting for censoring and temporal dependencies in the input data. In addition, an NN architecture (e.g., an attention mechanism or similar architecture) can be used to focus on critical time points or features to improve the survival model’s accuracy and interpretability. In preferred embodiments, the LOS Agent has a mean absolute error (MAE) of 2 or less, root mean squared error (RMSE) of 2.5 days or less, and/or R-Squared (R²) of 0.65 or more.

Turning to FIG. 5, a block diagram of a data processing system is depicted in accordance with an illustrative embodiment. Data processing system 500 is an example of a system in which computer-readable program code or program instructions implementing processes of illustrative embodiments may be run. Data processing system 500 may be an example of one system in which server 115, computing device 105, and/or computing device 110 in FIG. 1 may be implemented. In this illustrative example, data processing system 500 includes communications fabric 502, which provides communications between processor unit 504, memory 506, persistent storage 508, communications unit 510, input/output unit 512, and display 514.

Processor unit 504 serves to execute instructions for software applications and programs that may be loaded into memory 506. Processor unit 504 may be a set of one or more hardware processor devices or may be a multi-processor core, depending on the particular implementation. Further, processor unit 504 may be implemented using one or more heterogeneous processor systems, in which a main processor is present with secondary processors on a single chip. As another illustrative example, processor unit 504 may be a symmetric multi-processor system containing multiple processors of the same type.

A computer-readable storage device is any piece of hardware that is capable of storing information, such as, for example, without limitation, data, computer-readable program code in functional form, and/or other suitable information either on a transient basis and/or a persistent basis. Further, a computer-readable storage device excludes a propagation medium. Memory 506, in these examples, may be, for example, a random-access memory, or any other suitable volatile or non-volatile storage device. Persistent storage 508 may take various forms, depending on the particular implementation. For example, persistent storage 508 may contain one or more devices. For example, persistent storage 508 may be a hard drive, a flash memory, a rewritable optical disk, a rewritable magnetic tape, or some combination of the above. The media used by persistent storage 508 may be removable. For example, a removable hard drive may be used for persistent storage 508.

Communications unit 510, in this example, provides for communication with other computers, data processing systems, and devices via network communications unit 510 may provide communications using both physical and wireless communications links. The physical communications link may utilize, for example, a wire, cable, universal serial bus, or any other physical technology to establish a physical communications link for data processing system 500. The wireless communications link may utilize, for example, shortwave, high frequency, ultra-high frequency, microwave, wireless fidelity (Wi-Fi), Bluetooth technology, global system for mobile communications (GSM), code division multiple access (CDMA), second-generation (2G), third-generation (3G), fourth-generation (4G), 4G Long Term Evolution (LTE), LTE Advanced, or any other wireless communication technology or standard to establish a wireless communications link for data processing system 500.

Input/output unit 512 allows for the input and output of data with other devices that may be connected to data processing system 500. For example, input/output unit 512 may provide a connection for user input through a keypad, keyboard, and/or some other suitable input device. Display 514 provides a mechanism to display information to a user and may include touch screen capabilities to allow the user to make on-screen selections through user interfaces or input data, for example.

Instructions for the operating system, applications, and/or programs may be located in storage devices 516, which are in communication with processor unit 504 through communications fabric 502. In this illustrative example, the instructions are in a functional form on persistent storage 508. These instructions may be loaded into memory 506 for running by processor unit 504. The processes of the different embodiments may be performed by processor unit 504 using computer-implemented program instructions, which may be located in a memory, such as memory 506. These program instructions are referred to as program code, computer-usable program code, or computer-readable program code that may be read and run by a processor in processor unit 504. The program code, in the different embodiments, may be embodied on different physical computer-readable storage devices, such as memory 506 or persistent storage 508.

Program code 518 is located in a functional form on computer-readable media 520 that is selectively removable and may be loaded onto or transferred to data processing system 500 for running by processor unit 504. Program code 518 and computer-readable media 520 form computer program product 522. In one example, computer-readable media 520 may be computer-readable storage media 524 or computer-readable signal media 526. Computer-readable storage media 524 may include, for example, an optical or magnetic disc that is inserted or placed into a drive or other device that is part of persistent storage 508 for transfer onto a storage device, such as a hard drive, which is part of persistent storage 508. Computer-readable storage media 524 also may take the form of a persistent storage, such as a hard drive, a thumb drive, or a flash memory that is connected to data processing system 500. In some instances, computer-readable storage media 524 may not be removable from data processing system 500.

Alternatively, program code 518 may be transferred to data processing system 500 using computer-readable signal media 526. Computer-readable signal media 526 may be, for example, a propagated data signal containing program code 518. For example, computer-readable signal media 526 may be an electro-magnetic signal, an optical signal, and/or any other suitable type of signal. These signals may be transmitted over communication links, such as wireless communication links, an optical fiber cable, a coaxial cable, a wire, and/or any other suitable type of communications link. In other words, the communications link and/or the connection may be physical or wireless in the illustrative examples. The computer-readable media may also take the form of non-tangible media, such as communication links or wireless transmissions containing the program code.

In some illustrative embodiments, program code 518 may be downloaded over a network to persistent storage 508 from another device or data processing system through computer-readable signal media 526 for use within data processing system 500. For instance, program code stored in a computer-readable storage media in a data processing system may be downloaded over a network from the data processing system to data processing system 500. The data processing system providing program code 518 may be a server computer, a client computer, or some other device capable of storing and transmitting program code 518.

The different components illustrated for data processing system 500 are not meant to provide architectural limitations to the manner in which different embodiments may be implemented. The different illustrative embodiments may be implemented in a data processing system including components in addition to, or in place of, those illustrated for data processing system 500. Other components shown in FIG. 5 can be varied from the illustrative examples shown. The different embodiments may be implemented using any hardware device or system capable of executing program code. As one example, data processing system 500 may include organic components integrated with inorganic components and/or may be comprised entirely of organic components excluding a human being. For example, a storage device may be comprised of an organic semiconductor.

As another example, a computer-readable storage device in data processing system 500 is any hardware apparatus that may store data. Memory 506, persistent storage 508, and computer-readable storage media 524 are examples of physical storage devices in a tangible form.

In another example, a bus system may be used to implement communications fabric 502 and may be comprised of one or more buses, such as a system bus or an input/output bus. Of course, the bus system may be implemented using any suitable type of architecture that provides for a transfer of data between different components or devices attached to the bus system. Additionally, a communications unit may include one or more devices used to transmit and receive data, such as a modem or a network adapter. Further, a memory may be, for example, memory 506 or a cache such as found in an interface and memory controller hub that may be present in communications fabric 502.

The present invention may be a system, a method, and/or a computer program product. The computer program product may include a computer-readable storage medium or media having computer-readable program instructions thereon for causing a processor to carry out aspects of the instant disclosure.

The computer-readable storage medium can be a tangible device that can retain and store instructions for use by an instruction execution device. The computer-readable storage medium may be, for example, but is not limited to, an electronic storage device, a magnetic storage device, an optical storage device, an electromagnetic storage device, a semiconductor storage device, or any suitable combination of the foregoing.

A non-exhaustive list of more specific examples of the computer-readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer-readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer-readable program instructions by utilizing state information of the computer readable program instructions to personalize the electronic circuitry, in order to perform aspects of the present invention.

These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer program instructions may also be stored in a computer-readable medium that can direct a computer, other programmable data processing apparatus, or other devices to function in a particular manner, such that the instructions stored in the computer-readable medium produce an article of manufacture including instructions which implement the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

FIG. 6 illustrates an example deep neural network in which embodiments of the present technology may be implemented. The deep neural network (DNN) 600 includes an input layer 601, a plurality of hidden layers 602, and an output layer 603. In one embodiment, the DNN 600 is a deep auto-encoder neural network (deep ANN) or a convolutional neural network (CNN).

As illustrated, the DNN 600 has two hidden layers 602, although it is understood that alternative embodiments may have any number of two or more hidden layers. Each layer 601 to 603 may have one or more nodes (represented by circles in the diagrammatic network). As depicted by the connecting lines, each node in a current layer is connected to every other node in a previous layer and a next layer. This is referred to as a fully-connected neural network. Other neural network structures are also possible in alternative embodiments of the DNN 600, in which not every node in each layer is connected to every node in the previous and next layers.

Each node in the input layer 601 can be assigned a value and output that value to every node in the next layer (e.g., hidden layer). The nodes in the input layer 601 can represent features about a particular environment or setting. For example, a DNN 600 used for classifying whether an object is a rectangle may have an input node representing whether the object has flat edges. In this example, assigning a value of 1 to the node may represent that the object does have flat edges and assigning a value of 0 to the node may represent that the object does not have flat edges. In another example, a DNN 600 takes an image as input. In this case, the input nodes may each represent a pixel of the image, such as a pixel of a training image, where the assigned value may represent the intensity of the pixel. Following this example, an assigned value of 1 may indicate that the pixel is completely black and an assigned value of 0 may indicate that the pixel is completely white.

Each node in the hidden layers 602 can receive an outputted value from nodes in a previous layer (e.g., input layer) and associate each of the nodes in the previous layer with a weight. Each hidden node can then multiply each of the received values from the nodes in the previous layer with the weight associated with the nodes in the previous layer and output the sum of the products to each node in the next layer.

Nodes in the output layer 603 handle input values received from the nodes in the hidden layer 602 in a similar fashion. In one example, each output node in the output layer 603 may multiply each input value received from each node in the previous layer (e.g., hidden layer) with a weight and sum the products to generate an output value. The output value of each output node can output information in a predefined format, where the information has some relationship to the corresponding information from the previous layer. Example outputs may include, but are not limited to, classifications, relationships, measurements, instructions, and recommendations. For example, a DNN 600 that classifies whether the object is an ellipse, where an outputted value of 1 from the output node represents that the object is an ellipse and an outputted value of 0 represents that the object is not an ellipse. While the examples provided relate to classifying geometric shapes, this is only for illustrative purposes. The output nodes can also be used to classify any of a wide variety of objects and other features and otherwise output any of a wide variety of desired information in desired formats (e.g., scores).

Furthermore, it should be understood that embodiments discussed herein are not limited to the particular features and processing steps shown. The flowchart and diagrams in the figures illustrate the method and resulting architecture according to embodiments of the present disclosure. In this regard, each block in the flowchart or structural diagrams may represent a step or partial step, which comprise one or more procedures for implementing the illustrative embodiments. In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved.

The descriptions of the various embodiments of the instant disclosure have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method for scoring medical treatments, comprising:
training, by a computing device, a large concept model (LCM) based on labeled electronic health record (EHR) data and a selected training algorithm to generate a trained LCM;
determining, by the computing device, a first score representing a severity of an association between a medical condition and one or more a symptom or a biomarker of an EHR data using the trained LCM, the EHR data associated with a patient;
determining, by the computing device, a second score representing a severity of an association between a diagnostic test and the medical condition using the trained LCM;
determining, by the computing device, a third score representing a severity of an association between a medical treatment and the medical condition using the trained LCM; and
storing, in a non-transient data store, one or more of the first score, the second score, and the third score in association with the EHR data.

2. The method of claim 1, wherein the EHR data comprises information captured via a wearable computing device or a mobile device.

3. The method of claim 1, wherein the EHR data comprises information generated in real time.

4. The method of claim 1, further comprising:
generating, by the computing device, a report based on the EHR data using the trained LCM;
wherein the report comprises one or more of:
a consultation report;
a diagnostic report;
a discharge summary;
a medico-legal report; and
a progress report.

5. The method of claim 1, wherein the selected training algorithm comprises at least one of:
a backpropagation algorithm;
an optimization algorithm;
a forward propagation algorithm;
an initialization algorithm;
a regularization algorithm;
a gradient boosting tree algorithm; and
a masked language modeling algorithm.

6. The method of claim 1, wherein at least one of
the non-transient data store comprises a vector database; and
the EHR data is stored in a second vector database.

7. The method of claim 1, further comprising:
receiving, by the computing device, user authentication data;
determining, by the computing device, whether authenticated user data comprises the user authentication data; and
wherein determining the first score comprises:
determining, by the computing device, the first score when the authenticated user data comprises the user authentication data.

8. The method of claim 1, wherein
the EHR data comprises data generated via a speech-to-text algorithm.

9. The method of claim 1, further comprising:
generating, by the computing device, a user notification comprising one or more of:
an identified medical condition associated with the first score when the first score is at least a first predetermined value;
an identified diagnostic test associated with the second score when the second score is at least a second predetermined value; and
an identified medical treatment associated with the third score when the third score is at least a third predetermined value.

10. A computer program product, the computer program product comprising a computer readable storage medium having program code embodied therewith, the program code executable by a processor to:
train a large concept model (LCM) based on labeled electronic health record (EHR) data and a selected training algorithm to generate a trained LCM;
determine a first score representing a severity of an association between a medical condition and one or more a symptom or a biomarker of an EHR data using the trained LCM, the EHR data associated with a patient;
determine a second score representing a severity of an association between a diagnostic test and the medical condition using the trained LCM;
determine a third score representing a severity of an association between a medical treatment and the medical condition using the trained LCM; and
store, in a non-transient data store, one or more of the first score, the second score, and the third score in association with the EHR data.

11. The computer program product of claim 10, wherein the EHR data comprises information captured via a wearable computing device or a mobile device.

12. The computer program product of claim 10, wherein the EHR data comprises information generated in real time.

13. The computer program product of claim 10, wherein
the program code is further executable by the processor to generate a medical report based on the EHR data using the trained LCM;
the medical report comprises one or more of:
a consultation report;
a diagnostic report;
a discharge summary;
a medico-legal report; and
a progress report.

14. The computer program product of claim 10, wherein the selected training algorithm comprises at least one of:
a backpropagation algorithm;
an optimization algorithm;
a forward propagation algorithm;
an initialization algorithm;
a regularization algorithm;
a gradient boosting tree algorithm; and
a masked language modeling algorithm.

15. The computer program product of claim 10, wherein at least one of
the non-transient data store comprises a vector database and at least one of the first score, the second score, and the third score are stored in the vector database; and
the EHR data is stored in a second vector database.

16. The computer program product of claim 10, wherein the program code is further executable by the processor to:
receive user authentication data;
determine whether authenticated user data comprises the user authentication data; and
wherein program code to determine the third score comprises:
program code to determine the third score when the authenticated user data comprises the user authentication data.

17. The computer program product of claim 10, wherein the EHR data comprises data generated via a speech-to-text algorithm.

18. The computer program product of claim 10, wherein the program code is further executable by the processor to:
generate a user notification comprising one or more of:
an identified medical condition associated with the first score when the first score is at least a first predetermined value;
an identified diagnostic test associated with the second score when the second score is at least a second predetermined value; and
an identified medical treatment associated with the third score when the third score is at least a third predetermined value.

19. A method for scoring medical treatments, comprising:
determining a feature to extract from an electronic health record (EHR) data, the feature representing a medical parameter of the EHR data, the EHR data associated with a patient;
extracting the feature from the EHR data to form a feature data;
processing the feature data using a machine learning model (MLM) trained with labeled EHR data;
wherein
the labeled EHR data represents instances of one or more of a plurality of symptoms or a plurality of biomarkers each associated with at least one of a plurality of medical conditions, a plurality of diagnostic tests each associated with at least one of said medical conditions, and a plurality of medical treatments each associated with at least one of said medical conditions;
the labeled EHR data associating one or more of (i) each of a plurality of first scores with a severity of an indicative association between one of said medical conditions and at least one of said symptoms or at least one of said biomarkers, (ii) each of a plurality of second scores with a severity of an identifying association between one of said diagnostic tests and at least one of said medical conditions, and (iii) each of a plurality of third scores with a severity of a managerial association between one of said medical treatments and at least one of said medical conditions;
processing the feature data includes:
generating a semantic representation corresponding to the EHR data;
determining, based on the processing and the semantic representation, one or more of:
a first determined score corresponding to one of said first scores;
a second determined score corresponding to one of said second scores;
a third determined score corresponding to one of said third scores; and
storing, in a non-transient data store, one or more of the first determined score, the second determined score, and the third determined score in association with the EHR data.
